(19) United States
(12) Patent Application Publication
(10) Pub. No.: US 2025/0243132 A1
(43) Pub. Date: Jul. 31, 2025

(54) Systems and Processes for Catalytic Conversion of Bio-Based Materials

(71) Applicant: Gevo, Inc., Englewood, CO (US)

(72) Inventor: Jonathan Smith, Highlands Ranch, CO (US)

(21) Appl. No.: 19/042,497

(22) Filed: Jan. 31, 2025

Related U.S. Application Data
(60) Provisional application No. 63/627,425, filed on Jan. 31, 2024.

(51) Int. Cl. C07C 1/24; C07C 45/52

(52) U.S. Cl. CPC C07C 1/24; C07C 45/52; C07C 2529/06

(57) ABSTRACT

System and processes are provided for converting glycerol to acrolein, an aldehyde to isoprene, and one or more fusel oils to isoprene. Each of this processes utilize at least one catalyst that includes a zeolite and one or more metals dopants (e.g., Group 1A metal(s), Group 2A metals(s), or any combination thereof). The at least one catalyst can optionally include one or more non-metal dopants (e.g., boron, phosphor, or a combination thereof).

### CROSS-REFERENCE TO RELATED APPLICATIONS

This application claims priority to U.S. Provisional Patent Application No. 63/627,425 filed on Jan. 31, 2024, and entitled “Systems and Processes for Catalytic Conversion of C₁-C₅ Alcohols to Hydrocarbon Mixtures,” the disclosure of which is incorporated herein by reference in its entirety.

### TECHNICAL FIELD

Systems and processes for catalytic conversion of bio-based materials, and more specifically, for example, to catalytic processes resulting in the conversion of glycerol to acrolein and an aldehyde to isoprene are provided.

### BACKGROUND

There is an increasing demand for the use of biomass for partly replacing petroleum resources for the synthesis of fuels. The use of bioethanol for the synthesis of base stocks for fuels is therefore of great interest. The reaction at the root of the process of converting ethanol to a base stock for fuels is ethanol dehydration followed by ethylene oligomerization. Additionally, the valorization of other biomass feedstocks such as glycerol, butanediol, and one or more fusel oils co-produced during fermentations is of great interest to provide routes to bio-based chemical intermediates.

The most commonly used catalysts are high purity gamma aluminas, silica-aluminas, unprocessed zeolites (e.g., ZSM-5), zeolites modified by steaming, doped zeolites and other silica alumina based catalysts. However, there remains a need for improved, efficient, and cost effective catalytic processes resulting in the direct conversion of bio-based feedstocks to valuable chemical precursors.

### SUMMARY

Aspects of the current subject matter relate inter alia to systems and processes for converting bio-based materials to valuable chemical intermediates.

In one aspect, the present disclosure provides a process for converting glycerol to acrolein. The process includes contacting an input stream that includes glycerol with a catalyst in a reactor to form an output stream that includes acrolein. The catalyst includes a zeolite and one or more metal dopants, the one or more metal dopants including one or more first metal dopants, one or more second metal dopants, or both. In various aspects of the process, the reactor can be at a temperature from about 300° C. to about 450° C., a gauge pressure from 0.25 bar to about 2.0 bar, and a weight hourly space velocity (WHSV) from about 1.0 h⁻¹ to about 10.0 h⁻¹. In some aspects, the process also includes prior to contacting the input stream, adding the one or more metal dopants to the catalyst.

In some aspects of the process for converting glycerol to acrolein, the reactor can be a single bed reactor. In certain aspects, the single bed reactor can be a fixed bed reactor, a fluidized bed reactor, or a moving bed reactor.

In some aspects of the process for converting glycerol to acrolein, the glycerol can be present in the input stream in an amount that is from about 25 wt. % to about 99 wt. %. In certain aspects, the glycerol can be present in the input stream in an amount that is from about 75 wt. % to about 99 wt. %.

In some aspects of the process for converting glycerol to acrolein, the one or more metal dopants can be present in the catalyst in an amount from about 0.01 wt. % to about 2 wt. %. In certain aspects, the one or more metal dopants can be present in the catalyst in an amount of at least 0.05 wt. %.

In various aspects of the process for converting glycerol to acrolein, the catalyst also includes one or more non-metal dopants. In some aspects, the one or more non-metal dopants includes boron, phosphor, or a combination thereof. In some aspects, boron is present in the catalyst in an amount from about 0.01 wt. % to about 10 wt. %. In certain aspects, boron is present in the catalyst in an amount of at least 0.05 wt. %. In some aspects, phosphor is present in the catalyst in an amount from about 0.1 wt. % to about 10 wt. %. In certain aspects, phosphor is present in the catalyst in an amount of at least 1.5 wt. %.

In some aspects of the process for converting glycerol to acrolein, the zeolite can include a ZSM-5 zeolite. In some aspects, the zeolite can include a MFI zeolite, a BEA zeolite, a FER zeolite, a CHA zeolite, a FAU zeolite, or any combination thereof.

In some aspects of the process for converting glycerol to acrolein, the acrolein can be present in the output stream in an amount that is about 70 wt. % or greater. In some aspects, the acrolein can be present in the output stream in an amount from about 70 wt. % to about 85 wt. %. In certain aspects, the acrolein can be present in the output stream in an amount that does not exceed about 85 wt. %.

In various aspects of the process for converting glycerol to acrolein, the one or more first metal dopants can be present in the catalyst in an amount from about 0.01 wt. % to about 1.5 wt. %. In some aspects, the one or more first metal dopants can be present in the catalyst in an amount from about 0.01 wt. % to about 1.0 wt. %. In some aspects, the one or more first metal dopants can be present in the catalyst in an amount from about 0.05 wt. % to about 0.2 wt. %. In certain aspects, the one or more first metal dopants can be present in the catalyst in an amount that does not exceed about 1.5 wt. %.

In various aspects of the process for converting glycerol to acrolein, the one or more second metal dopants can be present in the catalyst in an amount from about 0.01 wt. % to about 1.5 wt. %. In some aspects, the one or more second metal dopants can be present in the catalyst in an amount from about 0.01 wt. % to about 1.0 wt. %. In some aspects, the one or more second metal dopants can be present in the catalyst in an amount from about 0.05 wt. % to about 0.2 wt. %. In certain aspects, the one or more second metal dopants can be present in the catalyst in an amount that does not exceed about 1.5 wt. %.

In some aspects of the process for converting glycerol to acrolein, the temperature can be from about 360° C. to about 400° C. In certain aspects, the temperature can be from about 360° C. to about 400° C. In some aspects, the WHSV can be from about 1.0 h⁻¹ to about 3.0 h⁻¹. In certain aspects, the WHSV can be from about 1.0 h⁻¹ to about 5.0 h⁻¹.

In some aspects of the process for converting glycerol to acrolein, the one or more first metal dopants can include sodium, potassium, lithium, or any combination thereof. In certain aspects, the one or more first metal dopants can include sodium. In some aspects, the one or more second metal dopants can include magnesium, calcium, strontium, barium, or any combination thereof.

In various aspects of the process for converting glycerol to acrolein, the catalyst also includes one or more other dopants. In some aspects, the one or more other dopants can include sulfur, scandium, yttrium, selenium, iron, manganese, tellurium, or any combination thereof. In some aspects, the one or more other dopants can be present in the catalyst in an amount from about 0.05 wt. % to about 1 wt. %. In some aspects, the one or more other dopants can be present in the catalyst at an amount from about 0.05 wt. % to about 0.5 wt. %. In certain aspects, the one or more other dopants can be present in the catalyst in an amount of at least 0.25 wt. %. In certain aspects, the one or more other dopants can be present in the catalyst in an amount that does not exceed about 2 wt. %.

In further aspects, the present disclosure provides a process for converting an aldehyde to isoprene. The process includes contacting an input stream including the aldehyde with a catalyst in a reactor to form an output stream that includes isoprene. The catalyst includes a zeolite and one or more metal dopants. The one or more metal dopants include one or more first metal dopants, one or more second metal dopants, or both. In various aspects of the process, the reactor can be at a temperature from about 300° C. to about 500° C., a gauge pressure from about 1.0 bar to about 5.0 bar, and a weight hourly space velocity (WHSV) from about 1.0 h⁻¹ to about 5.0 h⁻¹.

In some aspects of the process for converting an aldehyde to isoprene, the aldehyde can include C₅ aldehyde.

In some aspects of the process for converting an aldehyde to isoprene, the reactor can be a single bed reactor. In certain aspects, the single bed reactor can be a fixed bed reactor, a fluidized bed reactor, or a moving bed reactor.

In another aspect, the present disclosure provides a process for converting one or more fusel oils to isoprene. The process includes contacting an input stream including the one or more fusel oils with a first catalyst in a reactor to form a first stream that includes an aldehyde, and contacting the first stream with a second catalyst in a reactor to form an output stream that includes isoprene. In various aspects of the process, the second catalyst includes a zeolite and one or more metal dopants. In certain aspects, the one or more metal dopants include one or more first metal dopants, one or more second metal dopants, or both, wherein the first catalyst and the second catalyst are different. In various aspects of the process, the reactor can be at a temperature from about 300° C. to about 500° C., a gauge pressure from about 1.0 bar to about 5.0 bar, and a weight hourly space velocity (WHSV) from about 1.0 h⁻¹ to about 5.0 h⁻¹.

In some aspects of the process for converting one or more fusel oils to isoprene, the first catalyst can include zinc ZnO or ZrZnO.

In some aspects of the process for converting one or more fusel oils to isoprene, the aldehyde can be present in the first stream from about [] to about []. In certain aspects, the aldehyde can be present in the first stream from about [] to about [].

In some aspects of the process for converting one or more fusel oils to isoprene, the aldehyde can include a C₅ aldehyde. In certain aspects, the C₅ aldehyde can include 3-methylbutyraldehyde, 2-methylbutyraldehyde, or both. In further aspects, the C₅ aldehyde can be present in the first stream in an amount from about 45 wt. % to about 85% wt. %. In yet further aspects, the C₅ aldehyde can be present in the first stream in an amount from about 60 wt. % to about 85% wt. %. In certain aspects, the C₅ aldehyde can be present in the first stream in an amount that does not exceed about 85% wt. %.

In some aspects of the process for converting an aldehyde to isoprene and/or the process for converting one or more fusel oils to isoprene, the aldehyde can include 3-methylbutyraldehyde, 2-methylbutyraldehyde, or both.

In various aspects of the process for converting an aldehyde to isoprene and/or the process for converting one or more fusel oils to isoprene, the one or more metal dopants can be present in the catalyst or the second catalyst in an amount from about 0.01 wt. % to about 2 wt. %. In certain aspects, the one or more metal dopants can be present in the catalyst or the second catalyst in an amount of at least 0.15 wt. %.

In various aspects of the process for converting an aldehyde to isoprene and/or the process for converting fusel oil to isoprene, the catalyst or second catalyst can also include one or more non-metal dopants. In some aspects, the one or more non-metal dopants can include boron, phosphor, or a combination thereof. In some aspects, the boron can be present in the catalyst or the second catalyst in an amount from about 0.01 wt. % to about 10 wt. %. In certain aspects, the boron can be present in the catalyst or the second catalyst in an amount of at least 0.05 wt. %. In some aspects, the phosphor can be present in the catalyst or the second catalyst in an amount from about 0.1 wt. % to about 7 wt. %. In certain aspects, the phosphor can be present in the catalyst or the second catalyst in an amount of at least 1.5 wt. %.

In various aspects of the process for converting an aldehyde to isoprene and/or the process for converting one or more fusel oils to isoprene, the zeolite can include a ZSM-5 zeolite. In certain aspects, the zeolite can include a MFI zeolite, a BEA zeolite, a FER zeolite, a CHA zeolite, a FAU zeolite, or any combination thereof.

In various aspects of the process for converting an aldehyde to isoprene and/or the process for converting one or more fusel oils to isoprene, the isoprene can be present in the output stream in an amount that is about 65 wt. % or greater. In some aspects, the isoprene can be present in the output stream in an amount from about 65 wt. % to about 85 wt. %. In certain aspects, the isoprene can be present in the output stream in an amount that does not exceed about 85 wt. %.

In various aspects of the process for converting an aldehyde to isoprene and/or the process for converting one or more fusel oils to isoprene, the one or more first metal dopants can be present in the catalyst or the second catalyst in an amount from about 0.01 wt. % to about 1.0 wt. %. In some aspects, the one or more first metal dopants can be present in the catalyst or the second catalyst in an amount from about 0.3 wt. % to about 0.5 wt. %. In certain aspects, the one or more first metal dopants can be present in the catalyst or the second catalyst in an amount that does not exceed about 1.5 wt. %.

In various aspects of the process for converting an aldehyde to isoprene and/or the process for converting one or more fusel oils to isoprene, the one or more second metal dopants can be present in the catalyst or the second catalyst in an amount from about 0.1 wt. % to about 1.0 wt. %. In some aspects, the one or more second metal dopants can be present in the catalyst or the second catalyst in an amount from about 0.3 wt. % to about 0.5 wt. %. In certain aspects, the one or more second metal dopants can be present in the catalyst or the second catalyst in an amount that does not exceed about 1.5 wt. %.

In various aspects of the process for converting an aldehyde to isoprene and/or the process for converting one or more fusel oils to isoprene, the temperature can be from about 350° C. to about 425° C. In some aspects, the temperature can be from about 375° C. to about 425° C. In some aspects, the WHSV can be from about 1.0 h⁻¹ to about 3.0 h⁻¹. In some aspects, the WHSV can be from about 1.0 h⁻¹ to about 4.0 h⁻¹.

In various aspects of the process for converting an aldehyde to isoprene and/or the process for converting one or more fusel oils to isoprene, the one or more first metal dopants can include sodium. In some aspects, the one or more second metal dopants can include magnesium, calcium, strontium, barium, or any combination thereof.

In various aspects of the process for converting an aldehyde to isoprene and/or the process for converting one or more fusel oils to isoprene, the catalyst or the second catalyst can also include one or more other dopants. In some aspects, the one or more other dopants include sulfur, scandium, yttrium, selenium, iron, manganese, tellurium, or any combination thereof. In some aspects, the one or more other dopants can be present in the catalyst or the second catalyst in an amount from about 0.05 wt. % to about 2 wt. %. In some aspects, the one or more other dopants can be present in the catalyst or the second catalyst at an amount from about 0.05 wt. % to about 0.5 wt. %. In certain aspects, the one or more other dopants can be present in the catalyst in an amount of at least 0.25 wt. %. In certain aspects, the one or more other dopants can be present in the catalyst or the second catalyst in an amount that does not exceed about 2 wt. %.

In various aspects of the process for converting an aldehyde to isoprene and/or the process for converting one or more fusel oils to isoprene, the processes can also include prior to contacting the input stream, adding the one or more metal dopants to the catalyst or the second catalyst.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### DETAILED DESCRIPTION

In the following description, certain specific details are set forth in order to provide a thorough understanding of various aspects. However, one skilled in the art will understand that the disclosure may be practiced without these details. In other instances, well-known structures have not been shown or described in detail to avoid unnecessarily obscuring descriptions of the aspects. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

Reference throughout this specification to “one aspect” or “an aspect” means a particular feature, structure or characteristic described in connection with the aspect is included in at least one aspect. Thus, the appearances of the phrases “in one aspect” or “in an aspect” in various places throughout this specification are not necessarily all referring to the same aspect. Furthermore, the particular features, structures, or characteristics may be combined in any suitable manner in one or more aspects. Also, as used in this specification and the appended claims, the singular forms “a,” “an,” and “the” include plural referents unless the context clearly dictates otherwise. It should also be noted that the term “or” is generally employed in its sense including “and/or” unless the context clearly dictates otherwise.

The word “about” when immediately preceding a numerical value means a range of plus or minus 10% of that value, e.g., “about 50” means 45 to 55, “about 25,000” means 22,500 to 27,500, etc. Furthermore, the phrases “less than about” a value or “greater than about” a value should be understood in view of the definition of the term “about” provided herein.

“WHSV” refers to weight hourly space velocity and is defined as the weight of the feed flowing per unit weight of the catalyst per hour.

One or more fusel oils refer to a single fusel oil or a combination of two or more fusel oils. Fusel oil(s), also referred to as fusel alcohol(s) or fuselol, is/are mixtures of several higher alcohols (those with more than two carbons, chiefly amyl alcohol) produced as a by-product of ethanol and/or isobutanol fermentation and can be produced in concentrated form during the purification of ethanol.

All yields and conversions described herein are on a weight basis unless specified otherwise.

Aspects of the subject matter disclosed herein improve on earlier conversion approaches by, inter alia, providing processes in which Group 1A metal(s) (e.g., sodium, potassium, or lithium) and/or Group 2A metal(s) (e.g., magnesium, calcium, strontium, or barium) doped catalyst systems are used to convert (i) glycerol to acrolein and (ii) an aldehyde to isoprene in high yields at competitive costs. The doped catalyst systems described herein include at least a zeolite doped with one or more metals, where the one or more metals include Group 1A metal(s) and/or Group 2A metal(s). As a result, the processes described herein produce higher yields compared to processes that utilize non-doped catalyst systems.

In some aspects, the present disclosure provides a process for converting glycerol to acrolein. In general, such processes described herein include contacting an input stream that includes glycerol with a catalyst in a reactor to form an output stream that includes acrolein, where the catalyst includes a zeolite and one or more metal dopants, where the one or more metal dopants including one or more first metal dopants, one or more second metal dopants, or both. In various aspects of such processes, the reactor can be at a temperature from about 300° C. to about 450° C., including all the subranges in between, a gauge pressure from 0.25 bar to about 2.0 bar, including all the subranges in between, and a weight hourly space velocity (WHSV) from about 1.0 h⁻¹ to about 10.0 h⁻¹, including all the subranges in between. In some aspects, the process also includes prior to contacting the input stream, adding the one or more metal dopants to the catalyst. In certain aspects, the reactor can be a single bed reactor (e.g., a fixed bed reactor, a fluidized bed reactor, or a moving bed reactor).

Regarding the reactor for converting glycerol to acrolein, in some aspects, the reactor can be operated a temperature from about 350° C. to about 425° C., including all the subranges in between. In further aspects, the reactor can be operated at a temperature from about 360° C. to about 400° C., including all the subranges in between. In some aspects, the reactor can be operated at a gauge pressure from 0.25 bar to about 1.5 bar or from 1.0 bar to 2.0 bar, including all the subranges in between. In some aspects, the reactor can be operated at a WHSV from about 1.0 h⁻¹ to about 3.0 h⁻¹, including all the subranges in between. In some aspects, the reactor can be operated at a WHSV from about 1.0 h⁻¹ to about 1.5 h⁻¹, including all the subranges in between.

In some aspects of the present processes disclosed herein for converting glycerol to acrolein, the glycerol can be present in the input stream in an amount that is from about 25 wt. % to about 99 wt. %, including all the subranges in between. In certain aspects, the glycerol can be present in the input stream in an amount that is from about 75 wt. % to about 99 wt. %, from about 75 wt. % to about 85 wt. %, or from about 80 wt. % to about 95 wt. %, including all the subranges in between.

In some aspects of the present processes disclosed herein for converting glycerol to acrolein, the acrolein can be present in the output stream in an amount that is about 70 wt. % or greater. In some aspects, the acrolein can be present in the output stream in an amount from about 70 wt. % to about 85 wt. %, including all the subranges in between. In certain aspects, the acrolein can be present in the output stream in an amount that does not exceed about 85 wt. %.

In some aspects of the present processes disclosed herein for converting glycerol to acrolein, the one or more metals (e.g., Group 1A metal(s), Group 2A metal(s), or any combination thereof) can be present in the catalyst at a variety of different concentrations. In certain aspects, the one or more metals can be present in the catalyst in amount from about 0.01 wt. % to about 2 wt. %, including all the subranges in between. In one aspect, the one or more metal dopants are present in the catalyst in an amount of at least 0.05 wt. %.

In some aspects, the present disclosure provides a process for converting an aldehyde to isoprene. In general, such processes described herein includes contacting an input stream including the aldehyde with a catalyst in a reactor to form an output stream that includes isoprene, where the catalyst includes a zeolite and one or more metal dopants, and where the one or more metal dopants include one or more first metal dopants, one or more second metal dopants, or both. In various aspects of such processes, the reactor can be at a temperature from about 300° C. to about 500° C., including all the subranges in between, a gauge pressure from about 1.0 bar to about 5.0 bar, including all the subranges in between, and a weight hourly space velocity (WHSV) from about 1.0 h⁻¹ to about 5.0 h⁻¹, including all the subranges in between. In some aspects, the process also includes prior to contacting the input stream, adding the one or more metal dopants to the catalyst. In certain aspects, the reactor can be a single bed reactor (e.g., a fixed bed reactor, a fluidized bed reactor, or a moving bed reactor). In further aspects, the aldehyde can include C₅ aldehyde.

In some aspects, the present disclosure provides a process for converting one or more fusel oils to isoprene. In general, such processes includes contacting an input stream including the one or more fusel oils with a first catalyst in a reactor to form a first stream that includes an aldehyde, and contacting the first stream with a second catalyst in a reactor to form an output stream that includes isoprene, where the second catalyst includes a zeolite and one or more metal dopants, and where the one or more metal dopants include one or more first metal dopants, one or more second metal dopants, or both, wherein the first catalyst and the second catalyst are different. In various aspects of such processes, the reactor can be at a temperature from about 300° C. to about 500° C., including all the subranges in between, a gauge pressure from about 1.0 bar to about 5.0 bar, including all the subranges in between, and a weight hourly space velocity (WHSV) from about 1.0 h⁻¹ to about 5.0 h⁻¹, including all the subranges in between. In certain aspects, the process also includes prior to contacting the input stream, adding the one or more metal dopants to the second catalyst. In further aspects, the first catalyst can include zinc ZnO, or ZrZnO.

In some aspects of the process for converting one or more fusel oils to isoprene, the aldehyde can include a C₅ aldehyde. In certain aspects, the C₅ aldehyde can include 3-methylbutyraldehyde, 2-methylbutyraldehyde, or both. In further aspects, the C₅ aldehyde can be present in the first stream in an amount from about 45 wt. % to about 85% wt. %, including all the subranges in between. In yet further aspects, the C₅ aldehyde can be present in the first stream in an amount from about 60 wt. % to about 85% wt. %, including all the subranges in between. In certain aspects, the C₅ aldehyde can be present in the first stream in an amount that does not exceed about 85% wt. %, including all the subranges in between.

Regarding the reactor for converting an aldehyde to isoprene and for converting one or more fusel oils to isoprene, in some aspects, the reactor can be operated a temperature from about 350° C. to about 425° C., including all the subranges in between. In further aspects, the reactor can be operated at a temperature from about 375° C. to about 425° C., including all the subranges in between. In some aspects, the reactor can be operated at a gauge pressure from 1.5 bar to about 4.0 bar or from 1.0 bar to 2.0 bar, including all the subranges in between. In some aspects, the reactor can be operated at a WHSV from about 1.0 h⁻¹ to about 3.0 h⁻¹, including all the subranges in between. In some aspects, the reactor can be operated at a WHSV from about 1.0 h⁻¹ to about 4.0 h⁻¹, including all the subranges in between.

In some aspects of the present processes disclosed herein for converting an aldehyde to isoprene and for converting one or more fusel oils to isoprene, the isoprene can be present in the output stream in an amount that is about 65 wt. % or greater. In certain aspects, the isoprene can be present in the output stream in an amount from about 65 wt. % to about 85 wt. %, including all the subranges in between. In one aspect, the isoprene can be present in the output stream in an amount that does not exceed about 85 wt. %.

In some aspects of the present processes disclosed herein for converting an aldehyde to isoprene and for converting one or more fusel oils to isoprene, the one or more metals (e.g., Group 1A metal(s), Group 2A metal(s), or any combination thereof) can be present in the catalyst or the second catalyst at a variety of different concentrations. In some aspects, the one or more metals can be present in the catalyst or the second catalyst in amount from about 0.01 wt. % to about 2 wt. %, including all the subranges in between. In one aspect, the one or more metal dopants are present in the catalyst or second catalyst in an amount of at least 0.15 wt. %.

Unless indicated otherwise, the term “catalyst” as used herein (e.g., the catalyst used in the glycerol to acrolein conversion processes and in the aldehydes to isoprene conversion processes) and “second catalyst” as used herein (e.g., the second catalyst used in the one or more fusel oils to isoprene conversion processes) are used interchangeably. As such, unless indicated otherwise, the disclosures herein regarding “catalyst” also apply to “second catalyst.”

Non-limiting examples of suitable zeolites for the processes described herein include crystalline silicates of the group ZSM-5 (MFI framework), BEA, CHA, FER, FAU, MWW, MOR, EUO, MFS, ZSM-48, MTT or TON having Si/Al higher than 10, or a dealuminated crystalline silicate of the group ZSM5 (MFI or BEA frameworks), CHA, FER, FAU, MWW, MOR, EUO, MES, ZSM-48, MTT or TON having Si/Al higher than 10, or molecular sieves of the type silico-aluminophosphate of the group AEL. In certain aspects, the zeolite can be a ZSM-5 zeolite. In further aspects, the zeolite can be a MFI zeolite, a BEA zeolite, a FER zeolite, a CHA zeolite, a FAU zeolite, or any combination thereof.

Non-limiting examples of one or more metal dopants (e.g., Group 1A and/or Group 2A metal dopants) include sodium, potassium, lithium, beryllium, magnesium, calcium, strontium, barium, radium, or any combination thereof. In aspects, the one or more metal dopants include one or more first metal dopants, one or more second metal dopants, or any combination thereof. In certain aspects, the one or more first metal dopants can include Group 1A metal(s), such as sodium, lithium, potassium, or any combination thereof. In one aspect, the one or more first metal dopants include only sodium. In certain aspects, the one or second metal dopants can include a Group 2A metal, such as magnesium, calcium, strontium, barium, or any combination thereof.

In some aspects, the one or more first metal dopants are present in the catalyst in an amount from about 0.01 wt. % to about 1.5 wt. %, including all the subranges in between. In certain aspects, the one or more first metal dopants can be present in the catalyst in an amount from about 0.01 wt. % to about 1.0 wt. %, from about 0.05 wt. % to about 0.2 wt. %., or from 0.3 wt. % to about 0.5 wt. %, including all the subranges in between. In one aspect, the one or more first metal dopants can be present in the catalyst in an amount that does not exceed about 1.5 wt. %.

In some aspects, the one or more second metal dopants are present in the catalyst in an amount from about 0.01 wt. % to about 1.5 wt. %, including all the subranges in between. In certain aspects, the one or more second metal dopants can be present in the catalyst in an amount from about 0.01 wt. % to about 1.0 wt. %, from about 0.05 wt. % to about 0.2 wt. %, or from 0.3 wt. % to about 0.5 wt. %, including all the subranges in between. In one aspect, the one or more second metal dopants can be present in the catalyst in an amount that does not exceed about 1.5 wt. %.

In some aspects, the zeolite can be doped with only one metal dopant. In one aspect, the zeolite is only doped with sodium. In another aspect, the zeolite is only doped with lithium. In another aspect, the zeolite is only doped with potassium.

The manufacture of zeolite types A, X, and Y is generally carried out by mixing and heating sodium aluminate and sodium silicate solutions, whereupon a sodium aluminosilicate gel is formed. The silicon oxide and aluminum oxide containing compounds pass into the liquid phase from which the zeolites are formed by crystallization. As such, the crude crystalline zeolite containing the original alkali metal may be subsequently converted to an intermediate ammonium form followed by calcination about 500° C. to about 550° C., to remove the ammonium counterion, thus yielding its' final hydrogen form. In the case of Group 1A metals (e.g., Na, K, and/or Li), the residual alkali metal cation content from the original zeolite synthesis, as described previously, should be in an amount that does not effectively inactivate the zeolite catalyst (e.g., less than or equal to 2000 ppm). In general, commercially produced hydrogen form zeolites (e.g., Clariant, Zeolyst, etc) typically have residual sodium levels of less than or equal to 0.05 wt. % (e.g., 500 ppm). Higher residual levels of sodium (e.g., >2500 ppm) and other Group 1A metals (e.g., Li, K, etc.) can severely deactivate the zeolite catalysts rendering them unacceptable for chemistries requiring highly acidic catalytic activity.

To this end, the inventors were surprised to discover that a subsequent impregnation of a doped zeolite (e.g., doped with boron and phosphor) with low levels of Group 1A metals (e.g., Na<2000 ppm, K<2000 ppm, and/or Li<1000 ppm) results in improved yields of acrolein and isoprene, as compared to a doped zeolite without subsequent Group 1A impregnation. The ability to minimize formation of by products, resulting in improved acrolein content from glycerol and in improved isoprene content from aldehydes, simplifies downstream separations of the enriched acrolein output streams or isoprene output streams.

In certain aspects, the catalyst can include a zeolite doped with one or more first metal dopants, one or more second metal dopants, or any combination and further with one or more non-metal dopants. Non-limiting examples of non-metal dopants include boron, phosphor, or a combination thereof. In some aspects, the one or more non-metal dopants only include boron and phosphor. In further aspects, the one or more non-metal dopants include only phosphor.

In some aspects, the catalyst can include a zeolite doped with sodium, lithium, potassium, or any combination thereof and boron, phosphor, or both. In one aspect, the catalyst can include a zeolite doped with sodium, boron, and phosphor. In further aspects, the catalyst can include a zeolite doped with lithium, boron, and phosphor. In yet further aspects, the catalyst can include a zeolite doped with potassium, boron and phosphor. In any of the foregoing aspects, the zeolite can be a ZSM-5 zeolite.

Boron and phosphor can be present in the catalysts in a variety of different amounts. In some aspects, the boron can be present in the catalyst in amount from about 0.01 wt. % to about 10 wt. %, including all the subranges in between. In certain aspects, the boron can be present in the catalyst in an amount from about 0.05 wt. % to about 5 wt. %, including all the subranges in between. In certain aspects, the boron can be present in the catalyst in an amount from about 0.05 wt. % to about 3 wt. %, including all the subranges in between. In one aspect, the boron can be present in the catalyst in an amount of at least 0.05 wt. %. In some aspects, the phosphor can be present in the catalyst in amount from about 0.1 wt. % to about 7 wt. %, including all the subranges in between. In certain aspects, the phosphor can be present in the catalyst in an amount from about 0.01 wt. % to about 10 wt. %, including all subranges in between. In further aspects, the phosphor can be present in the catalyst in an amount from about 1.5 wt. % to about 6 wt. % or from about 0.1 wt. % to about 7 wt. %, including all the subranges in between. In one aspect, the phosphor can be present in the catalyst in an amount of at least 1.5 wt. %. In certain aspects, the boron can be present in the catalyst in amount from about 0.5 wt. % to about 3 wt. %, including all the subranges in between, and the phosphor can be present in the catalyst in an amount from about 1.5 wt. % to 6 wt. %, including all the subranges in between.

In some aspects, the catalysts disclosed herein can include a zeolite doped with boron and phosphor and a Group 1A or 2A metal, or a zeolite doped with a Group 1A or 2A metal. Without being bound by theory, it is believed the presence of boron increases the stability of the phosphor within the zeolite framework resulting in extended time on stream (TOS), while also maintaining selectivity. Further, without being bound by theory, it is believed that the presence of one or more Group 1A metals and/or one or more Group 2A metals (e.g., at between about 0.03-1.0 wt. % of the catalyst) in combination with boron/phosphor dopants within the zeolite additionally increases the stability of the boron/phosphor zeolite resulting in extended time on stream (TOS), while also maintaining selectivity due to the additional neutralization of residual strong acid sites within the zeolite framework not modified by the initial boron and/or phosphor impregnation. The ability to effectively titrate residual strong acid sites within the zeolite framework, not initially modified by impregnation with boron and phosphor, improves selectivity to acrolein and isoprene. Furthermore, the strong acid site titration may be accomplished by the simultaneous co-impregnation of boron, phosphor, and the Group 1A and/or Group 2A metal(s). The presence of one or more Group 1A metals and/or one or more Group 2A metals in such instances can further improve yields to acrolein and isoprene as compared to just a boron and/or phosphor doped zeolite.

In some aspects, the catalysts disclosed herein can be further doped with additional dopants, also referred to herein as other dopants, with or without the non-metal dopants. In one aspect, the catalyst can be doped with one or more metal dopants, one or more non-metal dopants, and one or more additional dopants. In further aspects, the catalyst can be doped with one or more metal dopants and one or more other dopants. Non-limiting examples of additional dopants include sulfur, scandium, yttrium, selenium, iron, manganese, tellurium, or any combination thereof.

In some aspects, the one or more other dopants can be present in the catalyst in an amount from about 0.05 wt. % to 2 wt. % or from about 0.05 wt. % to about 1 wt. %, including all the subranges in between. In further aspects, the one or more other dopants can be present in the catalyst at an amount from about 0.05 wt. % to about 0.5 wt. %, including all the subranges in between. In yet further aspects, the one or more other dopants can be present in the catalyst in an amount of at least 0.25 wt. %. In certain aspects, the one or more other dopants can be present in the catalyst in an amount that does not exceed about 2 wt. %.

Granular or extruded catalyst(s) can be used for the reactions described herein. For example, in some aspects, granular or extruded catalyst(s) can have a particle size of greater than at least 0.05 mm, about 0.1 mm or greater, or from about 0.05 mm to about 2.5 mm, including all the subranges in between. In one aspect, granular or extruded catalysts(s) can have a particle size from about 0.4 to about 2.0 mm, including all the subranges in between.

In some aspects, the process can include, after contacting the input stream or first stream with the catalyst in the reactor, regenerating the catalyst. In some aspects, the regeneration of the catalyst can be carried out by purging any gaseous or liquid hydrocarbons or oxygenates from the reactor and then introducing air and/or oxygen optionally diluted with inert gas or steam to combust any solid carbon deposits on the catalyst. In some aspects, the process can include, a system whereby the catalyst is circulated between a reactor in which it is contacted with the input stream or first stream and a regeneration reactor in which is it contacted with air and/or oxygen optionally diluted with inert gas or steam to combust any solid carbon deposits on the catalyst.

The following specific examples are intended to be illustrative of the invention and should not be construed as limiting the scope of the invention as defined by appended claims.

### EXAMPLES

### Example 1: Reactor Set-Up

Glycerol conversion to acrolein or one or more fusel oils to isoprene was carried out at 200° C.-500° C., via fixed bed reactors, containing specified catalyst(s), and flowing preheated (160° C.) vaporized feedstock in a downward flow over the fixed catalyst bed while co-feeding nitrogen at atmospheric pressure or under moderate pressures (i.e., 0-30 bar). The flow rate of feedstock was controlled by Teledyne Model 500D syringe pumps, and the flow rates were adjusted to obtain the targeted olefin WHSV (weight hourly space velocity). The internal reaction temperature was maintained constant via a Lindberg Blue M furnace as manufactured by Thermo-Scientific.

### Example 2: Impregnated Boron/Phosphor and Sodium (1.5% Boron, 3% Phosphor, 0.13% Sodium) Impregnated ZSM-5 Zeolite Catalyst Preparation

Boron, Phosphor, and Sodium impregnated zeolite catalyst was prepared by incipient wetness technique: 0.57 g phosphoric acid (85%), 0.43 g boric acid (99+%), and 0.0238 g sodium nitrate was dissolved in deionized water (3.7 mL). Upon heating and dissolution, the solution was added in dropwise fashion to 5 g ZSM-5 zeolite support (i.e., Zeolyst type CBV-5524 H+). The resulting impregnated catalyst was dried at 160° C. for 1 hr, and afterwards calcined at 550° C. for 3-15 hrs.

### Example 3: Single Stage Reactor (Prophetic Example)

Fixed Bed Catalyst—Step 1: Na-Doped ZSM-5 zeolite as described in Example 2 can be loaded into a single fixed bed reactor (2.9 g catalyst). The process conditions can be as follows: Glycerol feed rate (35 wt. % refined glycerol in water)=0.29 ml/min; T=375° C.; P=atm; N₂=10 cc/min; and TOS=21 h. Upon completion of the process, without being bound by theory, the isolated mass yield of acrolein can be equal to or greater than 85% based on 100% glycerol conversion, and the mass accountability can be greater than 95% (Mass out/Mass in).

Further, upon completion of the process, without being bound by theory, the component yield (wt. %) of the output stream can be:
Acetaldehyde: 4.2 wt. % of the output stream
Acrolein: 87.2 wt. % of the output stream
Propanal: 2.2 wt. % of the output stream
Acetol: 5.5 wt. % of the output stream
Other minor components not quantified: ethanol, diacetyl, allyl alcohol, and phenol

### Comparative Example 4: Single Stage Reactor

Fixed Bed Catalyst-Step 1: Boron/Phosphor doped ZSM5 zeolite as described in Example 2 without addition of Na was loaded into the single fixed bed reactor (2.9 g catalyst); Glycerol feed rate (35 wt % refined glycerol in water)=0.29 ml/min; T=375° C.; P=atm; N₂=10 cc/min. Isolated Mass Yield of Acrolein=>70% based on 100% glycerol conversion; TOS=21 h; Mass accountability>95% (Mass out/Mass in).

Upon completion of the process, the component yield (wt. %) of the output stream was as follows:
Acetaldehyde: 6.2 wt. % of output stream
Acrolein: 75.2 wt. % of the output stream
Propanal: 2.2 wt. % of the output stream
Acetol: 13.5 wt. % of the output stream
Other minor components not quantified: ethanol, diacetyl, allyl alcohol, and phenol

### Example 5: Single Stage Reactor

Single Stage reactor configuration: Dehydrogenation Reaction Conditions: T=460° C. in reactor, WHSV=1.8 (Crude Fusel Oil basis), Crude Fusel Oil Feed Composition: 6 wt % EtOH, 0.63% 1-propanol, 5.5% isobutanol, 0.57% 1-butanol, 54.5% 3-methyl-1-butanol, 18.9% 2-methyl-1-butanol, 12.6% water; P=0 bar; Catalyst—5% Zr-doped ZnO.

TABLE 1
Single pass reactor effluent composition and corresponding
weight percent of total. Bases: 95% conversion
of 3-methylbutanol/2-methylbutanol.

| Single Pass Reactor | Wt % of |
|---|---|
| Effluent Composition | Total: |
| Acetaldehyde | 1 |
| isobutyraldehyde | 4.4 |
| 3-methylbutyraldehyde | 51.6 |
| 2-methylbutyraldehyde | 17.7 |
| MIPK | 2.4 |
| 3/2-methyl-butanol | 5.8 |
| ketones | 6 |
| acids | 5 |
| esters | 4.8 |

### Example 6: Single Stage Reactor (Prophetic Example)

Single Stage reactor configuration: The reaction conditions of the process can be: T=390° C. in reactor, WHSV=2.4; P=0 bar. The input stream can be 70 wt %/30 wt % 3-methylbutyraldehyde/2-methylbutyraldehyde. Boron/Phosphor doped ZSM-5 zeolite as described in Example 2 with Na dopant can be loaded into the single fixed bed reactor (2.9 g catalyst).

Upon completion of the process, without being bound by theory, the single pass reactor effluent composition (output stream) and corresponding weight percent of total can be as follows as set forth in Table 2 below.

TABLE 2

| Single Pass Reactor | Wt. % of |
|---|---|
| Effluent Composition | Total: |
| isobutylene | 5.7 |
| C₅ olefins | 0.6 |
| Isoprene | 35.5 |
| 3-methyl-butyraldehyde | 45.0 |
| 2-methyl-butyraldehyde | 2.5 |
| MIPK | 1.1 |
| Pentanones | 2.4 |

### Comparative Example 7: Single Stage Reactor

Single Stage reactor configuration: Reaction Conditions: T=390° C. in reactor, WHSV=2.4; P=0 bar. The input stream included 70 wt %/30 wt % 3-methylbutyraldehyde/2-methylbutyraldehyde . . . Boron/Phosphor doped ZSM5 zeolite as described in Example 2 without Na dopant was loaded into the single fixed bed reactor (2.9 g catalyst).

TABLE 3
Single pass reactor effluent composition and
corresponding weight percent of total.

| Single Pass Reactor | Wt % of |
|---|---|
| Effluent Composition | Total: |
| isobutylene | 5.7 |
| C₅ olefins | 1.6 |
| Isoprene | 16.5 |
| 3-methyl-butyraldehyde | 55.0 |
| 2-methyl-butyraldehyde | 10.5 |
| MIPK | 1.1 |
| Pentanones | 4.4 |

It is understood that the examples and aspects described herein are for illustrative purposes only and that various modifications or changes in light thereof will be suggested to persons skilled in the art and are to be included within the spirit and purview of this application and scope of the appended claims.

Although various illustrative aspects are described above, any of a number of changes can be made to various aspects without departing from the teachings herein. For example, the order in which various described method steps are performed can often be changed in alternative aspects, and in other alternative aspects, one or more method steps can be skipped altogether. Optional features of various system and process aspects can be included in some aspects and not in others. Therefore, the foregoing description is provided primarily for exemplary purposes and should not be interpreted to limit the scope of the appended claims.

The examples and illustrations included herein show, by way of illustration and not of limitation, specific aspects in which the subject matter can be practiced. As mentioned, other aspects can be utilized and derived there from, such that structural and logical substitutions and changes can be made without departing from the scope of this disclosure. Such aspects of the inventive subject matter can be referred to herein individually or collectively by the term “invention” merely for convenience and without intending to voluntarily limit the scope of this application to any single invention or inventive concept, if more than one is, in fact, disclosed. Thus, although specific aspects have been illustrated and described herein, any arrangement calculated to achieve the same purpose can be substituted for the specific aspects shown. This disclosure is intended to cover any and all adaptations or variations of various aspects. Combinations of the above aspects, and other aspects not specifically described herein, will be apparent to those of skill in the art upon reviewing the above description. Use of the term “based on,” herein and in the claims is intended to mean, “based at least in part on,” such that an unrecited feature or element is also permissible.

The subject matter described herein can be embodied in systems and methods depending on the desired configuration. The aspects set forth in the foregoing description do not represent all aspects consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the aspects described herein can be directed to various combinations and subcombinations of the disclosed features and/or combinations and subcombinations of several further features disclosed herein. Other aspects can be within the scope of the appended claims.

1. A process for converting glycerol to acrolein, the process comprising:
contacting an input stream comprising glycerol with a catalyst in a reactor to form an output stream comprising acrolein, the catalyst comprising a zeolite and one or more metal dopants, the one or more metal dopants comprise one or more first metal dopants, one or more second metal dopants, or both;
wherein the reactor is at a temperature from about 300° C. to about 450° C., a gauge pressure from 0.25 bar to about 2.0 bar, and a weight hourly space velocity (WHSV) from about 1.0 h⁻¹ to about 10.0 h⁻¹.

2-39. (canceled)

40. A process for converting an aldehyde to isoprene, the process comprising:
contacting an input stream comprising the aldehyde with a catalyst in a reactor to form an output stream comprising isoprene, the catalyst comprising a zeolite and one or more metal dopants, the one or more metal dopants comprise one or more first metal dopants, one or more second metal dopants, or both;
wherein the reactor is at a temperature from about 300° C. to about 500° C., a gauge pressure from about 1.0 bar to about 5.0 bar, and a weight hourly space velocity (WHSV) from about 1.0 h⁻¹ to about 5.0 h⁻¹.

41-43. (canceled)

44. A process for converting one or more fusel oils to isoprene, the process comprising:
contacting an input stream comprising the one or more fusel oils with a first catalyst in a reactor to form a first stream comprising an aldehyde;
contacting the first stream with a second catalyst in a reactor to form an output stream comprising isoprene, the second catalyst comprising a zeolite and one or more metal dopants, the one or more metal dopants comprise one or more first metal dopants, one or more second metal dopants, or both, wherein the first catalyst and the second catalyst are different;
wherein the reactor is at a temperature from about 300° C. to about 500° C., a gauge pressure from about 1.0 bar to about 5.0 bar, and a weight hourly space velocity (WHSV) from about 1.0 h⁻¹ to about 5.0 h⁻¹.

45-85. (canceled)
